(19) United States
(12) Patent Application Publication
(10) Pub. No.: US 2026/0174343 A1
(43) Pub. Date: Jun. 25, 2026

(54) CUFF AND BLOOD PRESSURE MEASUREMENT DEVICE

(71) Applicant: OMRON HEALTHCARE Co., Ltd., Kyoto (JP)

(72) Inventors: Wataru TSUNODA, Kyoto (JP); Daizo OKA, Kyoto (JP); Yuki OTA, Kyoto (JP); Naomi MATSUMURA, Kyoto (JP)

(21) Appl. No.: 19/545,686

(22) Filed: Feb. 20, 2026

Related U.S. Application Data
(63) Continuation of application No. PCT/JP2024/040031, filed on Nov. 11, 2024.

(30) Foreign Application Priority Data
Feb. 27, 2024 (JP) 2024-027674

(51) Int. Cl. A61B 5/022; A61B 90/00

(52) U.S. Cl. CPC A61B 5/02233; A61B 90/08; A61B 2090/0807

(57) ABSTRACT

A cuff to be wrapped and attached around a measurement target portion for blood pressure measurement, the cuff including: a bag-shaped exterior portion having a circumferential direction that is a winding direction around the measurement target portion and a width direction that is a direction orthogonal to the circumferential direction, and including an outer surface that is on an outer side and an inner surface that is on an inner side when the cuff is attached; a fluid bag accommodated in the exterior portion; and a surface fastener provided on a surface of the exterior portion, wherein a scale that divides the exterior portion in the circumferential direction is provided on the surface of the exterior portion, and the cuff is configured to be able to specify where a predetermined portion of the cuff is positioned on the scale when the cuff is attached.

### CROSS-REFERENCE TO RELATED APPLICATIONS

This application is the U.S. national stage application filed pursuant to 35 U.S.C. 365(c) and 120 as a continuation of International Patent Application No. PCT/JP2024/040031, filed Nov. 11, 2024, which application claims priority to Japanese Patent Application No. 2024-027674, filed Feb. 27, 2024, which applications are incorporated herein by reference in their entireties.

### FIELD

The present invention relates to the technical field of measuring blood pressure of a living body, and particularly to a cuff used for a blood pressure measurement device.

### BACKGROUND ART

In recent years, it has become widespread to perform health management by measuring information related to the body and health of an individual, such as a blood pressure value, with a measurement device and recording and analyzing the measurement results. There are provided various devices such as blood pressure measurement devices for home use so that the measurement of biological information can be easily performed outside medical institutions such as at home.

In a general blood pressure measurement device, a cuff for compressing a blood vessel is used to measure a blood pressure value by compressing the blood vessel and detecting a pulse wave propagating through the blood vessel. Specifically, a cuff is wrapped around a measurement site (for example, an upper arm), and then air is poured into the fluid bag inside the cuff to inflate the wrapped cuff, thereby compressing the blood vessel of the measurement site.

Therefore, various devices have been proposed for cuffs used for blood pressure measurement, depending on the purpose of facilitating the wrapping around the measurement site and reducing the discomfort during attachment (Patent Documents 1 and 2, etc.).

### CITATION LIST—PATENT LITERATURE

Patent Document 1: JP 2002-209858 A
Patent Document 2: JP 2014-018260 A

### SUMMARY OF INVENTION

### Technical Problem

In order to obtain a correct measurement value, it is necessary to correctly attach the cuff and perform measurement in a correct measurement posture. In addition, in continuously measuring blood pressure for health management, it is also important that conditions for daily blood pressure measurement do not vary (as little variation as possible).

That is, if the manner of attachment of the cuff (the strength of the wrapping, the orientation of the cuff, etc.) is different for each measurement, the conditions in each measurement are not constant, and not only is there a risk that accurate blood pressure measurement may not be possible, but the significance of continuously measuring the blood pressure value (accumulating data) is diminished. However, the known techniques including the above-described patent documents cannot assist the user in attaching the cuff in the same manner every time.

In view of the above circumstances, an object of the present invention is to provide a technique for enabling a cuff to be attached in an appropriate wrapping manner every time blood pressure measurement is performed.

### Solution to Problem

The present invention employs the following configurations to solve the above-described problems. That is, a cuff to be wrapped and attached around a measurement target portion for blood pressure measurement, the cuff including a bag-shaped exterior portion having a circumferential direction that is a winding direction around the measurement target portion and a width direction that is a direction orthogonal to the circumferential direction, and including an outer surface that is on an outer side and an inner surface that is on an inner side (a surface having a region that comes into contact with the measurement target portion when the cuff is wound) when the cuff is attached, a fluid bag accommodated in the exterior portion, and a surface fastener provided on a surface of the exterior portion, wherein a scale that divides the exterior portion in the circumferential direction is provided on the surface of the exterior portion, and the cuff is configured to be able to specify where a predetermined portion of the cuff is positioned on the scale when the cuff is attached.

The scale here does not necessarily have to be expressed by lines, and may function as markers that divide the circumferential length of the cuff into a plurality of stages, such as dots, symbols, figures, or characters. The marks of the scale do not necessarily indicate the actual length.

With this configuration, it is possible to provide a cuff that can be attached with the same degree of winding every time without adding a new component (at low cost).

The scale may be provided in a region including a central portion in the width direction, or may be provided in a region including at least one end portion in the width direction.

The scale may be provided in each of regions including both end portions in the width direction. The exterior portion may be provided with markers indicating positions corresponding to each of the scales.

For example, in the case of attaching the cuff to the upper arm, the length of the arm circumference is different (tapered) between both ends in the width direction of the cuff (that is, the upper side and the lower side of the arm), so that the degree of winding differs between both ends in the width direction to correctly attach the cuff. In this regard, with the configuration described above, since different appropriate scales can be provided at both ends of the cuff in the width direction, appropriate positioning can be performed at both ends in the width direction even when there is a taper.

The markers indicating the scale at one end portion in the width direction and the markers indicating the scale at the other end portion may be provided at different positions in the circumferential direction, reflecting a taper of the measurement target portion in the width direction. With this configuration, it is possible to present the scales indicating the same marks while reflecting the difference in the circumferential length at the time of winding at both ends in the width direction of the measurement target portion due to the taper.

The cuff may further include a fold-back ring for folding back through an outer end portion that is one end portion of the exterior portion in the circumferential direction and is exposed to the outside when the cuff is attached, the scale may be provided on the inner surface, and the predetermined portion may be a fold-back portion of the exterior portion when the cuff is attached.

With such a configuration, in a cuff of a type in which both the hook and the loop of the surface fastener are formed on the outer surface out of the inner surface and the outer surface of the exterior portion (folded-back type), the cuff is firmly fixed for appropriate attachment, and the positional relationship with the scale can be determined with reference to the position of the folded-back portion having a small winding error of the folded-back outer end portion.

The exterior portion may further accommodate a curler that has an elastic force and assists in maintaining the cuff in an annular shape, the scale may be provided on the outer surface, and the predetermined portion may be the circumferential end portion of the exterior portion that is exposed to the outside when the cuff is attached. That is, a scale indicating how much the exterior portion of the attached cuff is wound in two layers may be provided on the outer surface of the exterior portion.

Further, the exterior portion may accommodate a curler that has an elastic force and assists in maintaining the cuff in an annular shape, the scale may be provided on the outer surface, and the predetermined portion may be a marker provided on the outer surface of the exterior portion. One or a plurality of markers may be provided.

With such a configuration, even in a cuff of a type in which a curler is further accommodated in the exterior portion, the wrapped state of the cuff in an appropriate attachment state can be repeatedly reproduced.

The present invention can also be understood as a blood pressure measurement device including a cuff as described above and a sphygmomanometer body connected to the cuff.

Note that the configurations described above can be combined with one another to constitute the present invention unless the combination leads to technical contradiction.

### Advantageous Effects of Invention

According to the present invention, it is possible to provide a technique capable of attaching a cuff in an appropriate winding manner every time blood pressure is measured.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1(A) is a schematic plan view of an outer surface of a cuff according to Example 1. FIG. 1(B) is a schematic plan view of an inner surface of the cuff according to Example 1.

FIG. 2(A) is a first explanatory diagram illustrating a usage mode of the cuff according to Example 1. FIG. 2(B) is a schematic diagram illustrating an outline of a measure used together with the cuff according to the Example 1.

FIG. 3 is a schematic diagram illustrating a state in which the cuff of Example 1 is attached.

FIG. 4 is a schematic diagram illustrating a cuff according to a modification of Example 1.

FIG. 5 is a schematic diagram illustrating a state in which a cuff according to a modification of Example 1 is attached.

FIG. 6(A) is a schematic plan view of an outer surface of a cuff according to Example 2.

FIG. 6(B) is a schematic plan view of an inner surface of the cuff according to Example 2.

FIG. 7(A) is a schematic diagram of a curler used for the cuff according to Example 2.

FIG. 7(B) is a schematic diagram illustrating a state in which the cuff of Example 2 is attached.

FIG. 8(A) is a schematic plan view of an outer surface of a cuff according to Example 3.

FIG. 8(B) is a schematic plan view of an inner surface of the cuff according to Example 3.

FIG. 9 is a schematic diagram illustrating a state in which the cuff of Example 3 is attached.

FIG. 10(A) is a schematic plan view of an outer surface of a cuff according to Example 4.

FIG. 10(B) is a schematic plan view of an inner surface of the cuff according to Example 4.

FIG. 11 is a schematic diagram illustrating a state in which the cuff of Example 4 is attached.

### DESCRIPTION OF EMBODIMENTS

### Example 1

Examples of the present invention will be described below with reference to the drawings. Note that the material, shape, relative arrangement, and the like of configurations described in these examples are not intended to limit the scope of the present invention to the configurations alone, unless otherwise stated.

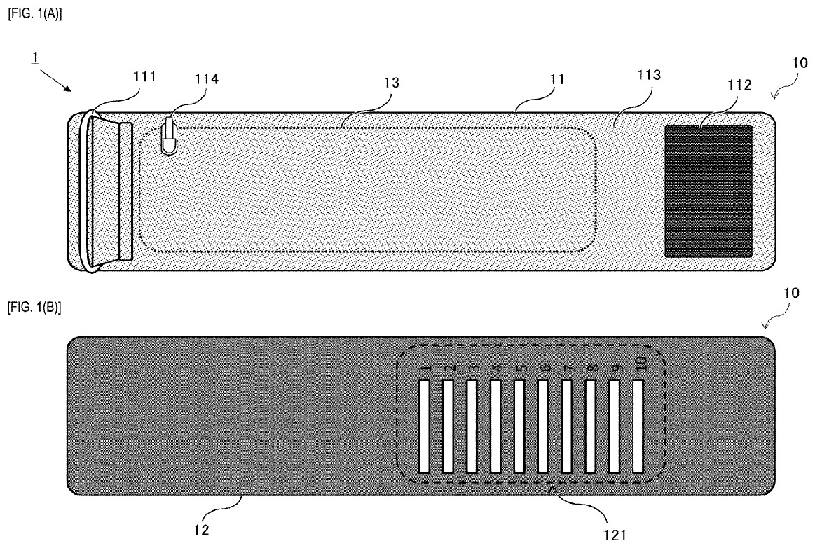

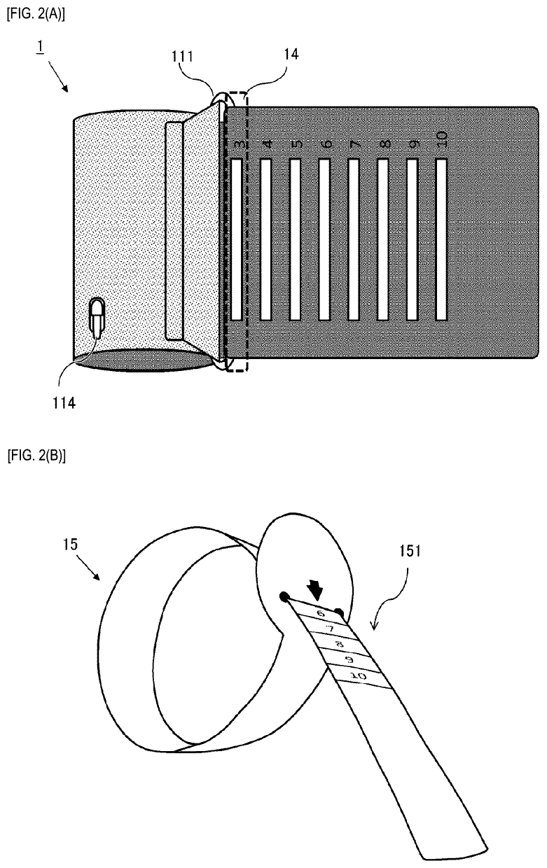

The present invention can be applied to a cuff 1 as illustrated in, for example, FIGS. 1(A), 1(B), and 2(A). FIG. 1(A) is a plan view of a surface (outer surface 11) of the cuff 1 that becomes an outer side when being attached, and FIG. 1(B) is a plan view of a surface (inner surface 12) of the cuff 1 that becomes an inner side when being attached. As illustrated in FIGS. 1(A) and 1(B), the cuff 1 has a bag-shaped exterior portion 10 having the outer surface 11 and the inner surface 12, and a fluid bag 13 (illustrated by the dotted line in FIG. 1(A)) is accommodated inside the exterior portion 10.

On the outer surface 11 of the exterior portion 10, a hook portion 112 and a loop portion 113 of a surface fastener composed of hooks and loops are formed. In the present example, the loop portion 113 of the surface fastener is formed over the entire region of the outer surface 11. A nipple 114 for taking air into and out of the fluid bag 13 is formed outside the exterior portion 10 illustrated in FIG. 1(A). The nipple 114 and a sphygmomanometer body including a pump are connected by a tube (both are not illustrated), thereby enabling air to be taken into and out of the fluid bag 13.

In addition, on the outer side of the exterior portion 10, a fold-back ring 111 through which the other end portion can be inserted is provided near one end portion in the circumferential direction (indicated by the left-right direction in FIG. 1(A)) that is a direction of being wound around the measurement portion at the time of blood pressure measurement. In attaching the cuff 1 to the measurement target portion, the other end portion of the exterior portion 10 is inserted through the fold-back ring 111 and is folded back, and the hook portion 112 and the loop portion 113 on the outer surface 11 are engaged with each other, whereby the cuff 1 is fixed to the measurement target portion.

On the other hand, on the inner surface 12 of the exterior portion 10, a scale 121 for dividing the cuff 1 in the circumferential direction is provided by printing, for example. As illustrated in FIG. 1(B), the scale 121 is indicated by a line extending in the width direction orthogonal to the circumferential direction, and is disposed so as to be exposed to the side (outside) visible to the user when the exterior portion 10 is folded back at the fold-back ring 111.

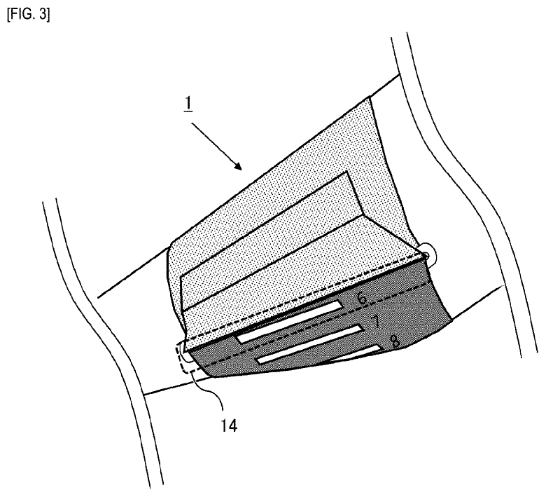

FIG. 2(A) is a diagram schematically illustrating a state in which the other end portion of the exterior portion 10 is inserted into the fold-back ring 111 and then is folded back. FIG. 3 is a diagram schematically illustrating a state in which the cuff 1 is attached to the upper arm of the user. As illustrated in FIG. 2(A) or FIG. 3, when the scale 121 is exposed to the outside, the scale 121 can indicate where a portion (fold-back portion 14) of the exterior portion 10 folded back by the fold-back ring 111 is positioned in the circumferential direction.

In attaching the cuff 1 to the measurement target portion, the user winds and fixes the cuff 1 such that the fold-back portion 14 comes at the same scale position, thereby preventing a situation in which the degree of winding of the cuff varies (tight or loose) every time blood pressure measurement is performed.

Since the proper way of winding (degree of winding) of the cuff 1 depends on the scale where the fold-back portion 14 is positioned and depends on the individual user, the user needs to grasp the correct relationship between the fold-back portion 14 and the corresponding scale. In this case, the circumferential length of the periphery of the measurement target portion (for example, the circumference of the upper arm) is measured with a dedicated measure formed of paper, a resin sheet, or the like, and the scale for positioning in attaching the cuff 1 can be obtained based on the measurement result. FIG. 2(B) illustrates such a dedicated measure 15.

The measure 15 is provided with a scale 151 corresponding to the scale 121 provided on the exterior portion 10 of the cuff 1. The scale of the cuff 1 corresponding to the value that is indicated by the black arrow illustrated in FIG. 2(B) when the measure 15 is wound around the measurement target portion (“6” in the example of FIG. 2(B)) is the scale indicating the position of the fold-back portion 14 when the measure 15 is wound in the correct manner. The user may measure the circumferential length of the measurement target portion by the measure 15 at an appropriate timing such as the first use of the cuff 1. The scale 151 of the measure 15 and the scale 121 provided on the cuff 1 do not coincide with each other, and the scale interval reflects the thickness of each scale.

In this way, the correct winding manner (winding degree) of the cuff 1 can be recognized and the correct winding manner can be repeatedly reproduced. This makes it possible to continuously acquire correct measurement data.

### Modification

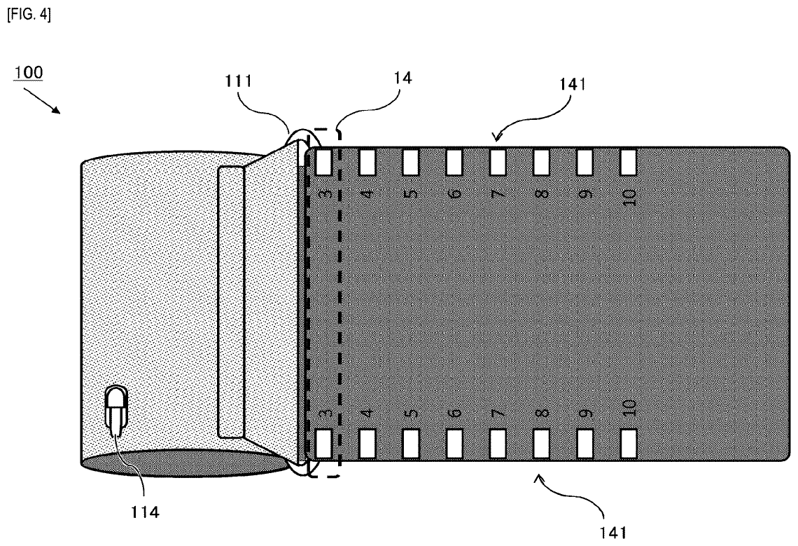

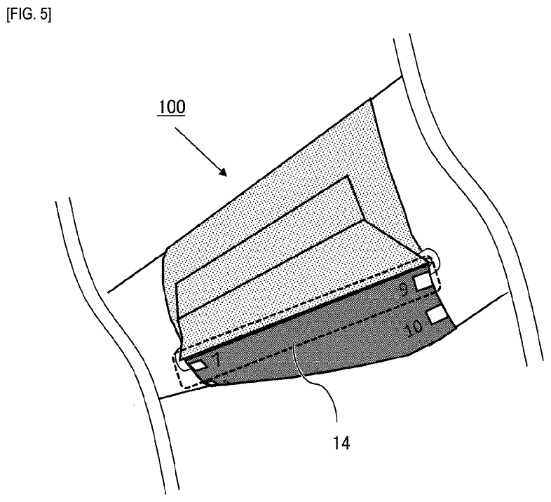

In Example 1 described above, each of the scales is provided so as to cross the width direction of the exterior portion 10 (that is, in a region including the central portion in the width direction). However, the scale is not limited to the scale shown in Example 1 described above. FIGS. 4 and 5 illustrate such a modification. In the following description, the same components as those already described are denoted by the same reference numerals, and detailed description thereof will be omitted. FIG. 4 is a diagram schematically illustrating a state in which one end portion of a cuff 100 according to the present modification is folded back by a fold-back ring 111, and FIG. 5 is a diagram schematically illustrating a state in which the cuff 100 is attached to the upper arm of the user.

As illustrated in FIG. 4, the cuff 100 according to the present modification has a configuration in which scales 141 are provided at both end portions in the width direction. As illustrated in FIG. 5, in a state where the cuff 100 is attached to the upper arm of the user, one scale and the other scale in the width direction of the fold-back portion 14 have different numerical values, reflecting the difference in the arm circumference between the side (upper side) close to the shoulder of the arm and the side (lower side) close to the wrist. In the example of FIG. 5, the scale of the end portion in the width direction of the cuff 100 positioned on the lower side of the arm has smaller numerical values than the scale positioned on the upper side.

In the present modification, it is desirable that the circumferential lengths of the measurement target portion are measured in advance by the measure 15 in accordance with the positions of the end portions of the cuff in the width direction. In this way, the fold-back portion 14 can be positioned in accordance with the appropriate scales in accordance with the difference in the circumferential length between the both end portions in the width direction of the cuff of the measurement target portion.

### Example 2

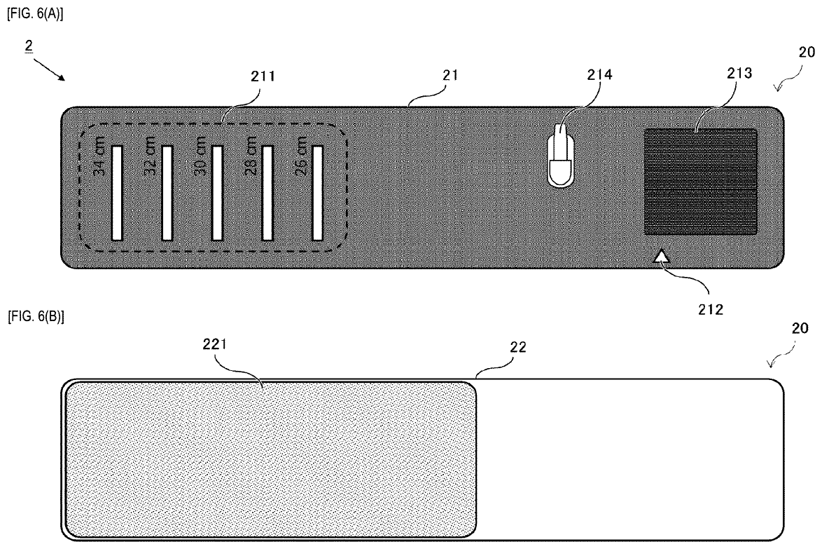

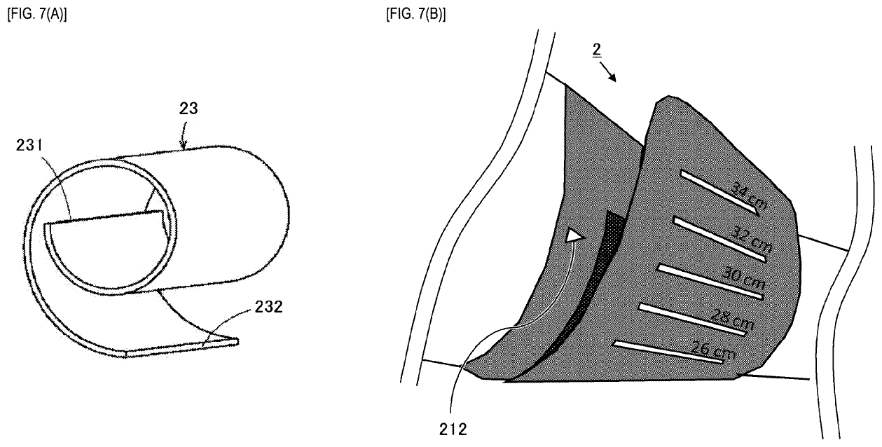

Next, a cuff 2 according to another example will be described with reference to FIGS. 6(A), 6(B), 7(A), and 7(B). FIG. 6(A) is a plan view of a surface (outer surface 21) of the cuff 2 that becomes an outer side when being attached, and FIG. 6(B) is a plan view of a surface (inner surface 22) of the cuff 2 that becomes an inner side when being attached. As illustrated in FIGS. 6(A) and 6(B), the cuff 2 is configured such that a fluid bag (not illustrated) and a curler 23 described later are accommodated in a bag-shaped exterior portion 20 having the outer surface 21 and the inner surface 22, similarly to the cuff 1 of Example 1.

A hook portion 213 of a surface fastener composed of hooks and loops is formed on the outer surface 21 of the exterior portion 20, and a loop portion 221 of the surface fastener is formed on the inner surface 22 of the exterior portion 20. Therefore, when the cuff 2 is attached, the hook portion 213 and the loop portion 221 are fixed by winding the cuff 2 so that the loop portion 221 covers the hook portion 213. A nipple 214 for taking air into and out of the fluid bag is formed outside the exterior portion 20.

The outer surface 21 of the exterior portion 20 is provided with a scale 211 and a marker 212 for indicating the scale. In the present example, indicators of the circumferential length of the measurement portion (for example, the upper arm) are also provided along with the scale.

The cuff 2 accommodates the curler 23 as illustrated in FIG. 7(A) on a side closer to the outer surface 21 than the fluid bag in the exterior portion 20. The curler 23 is formed of an elastic member such as a resin plate, and has a shape in which one end portion 231 is wound inward and the other end portion 232 is extended outward. Since the curler 23 is accommodated in the exterior portion 20, the cuff 2 is biased such that the portion where the curler 23 is accommodated is formed into an annular shape, and in attaching the cuff 2, the curler 23 supports the cuff 2 so as to be maintained in a wound state.

Next, the scale 211 and the marker 212 of the cuff 2 will be described with reference to FIG. 7(B). FIG. 7(B) is a schematic diagram illustrating a state in which the cuff 2 is attached to the upper arm of the user. As illustrated in FIG. 7(B), when the cuff 2 is attached to the upper arm, the end portion of the outer surface 21 on the side where the scale 211 is provided is disposed on the outermost side. Therefore, when the cuff 2 is attached, the marker 212 provided near the other end portion of the cuff 2 indicates the scale corresponding to the length of the arm circumference of the upper arm around which the cuff 2 is wound.

With a configuration as described above, even in the case of the cuff 2 of the type incorporating the curler 23, the user can attach the cuff 2 in the same winding manner (degree of winding) at all times by attaching the cuff 2 such that the positional relationship between the scale 211 and the marker 212 is always consistent.

### Example 3

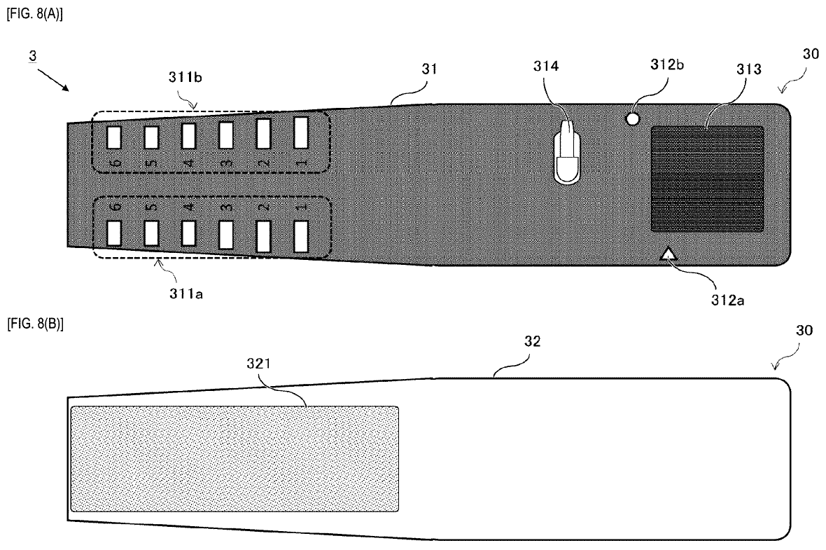

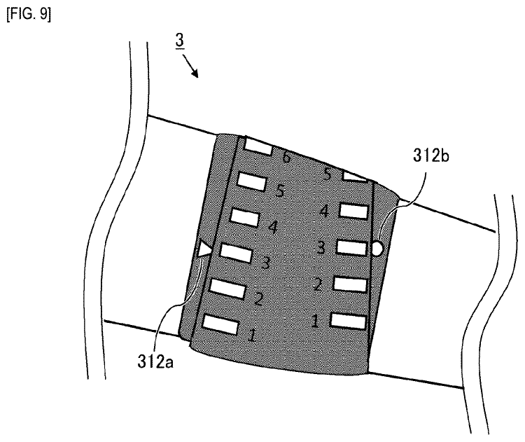

Next, a cuff 3 according to still another example will be described with reference to FIGS. 8(A), 8(B), and 9. FIG. 8(A) is a plan view of a surface (outer surface 31) of the cuff 3 that becomes an outer side when being attached, and FIG. 8(B) is a plan view of a surface (inner surface 32) of the cuff 3 that becomes an inner side when being attached. FIG. 9 is a diagram schematically illustrating a state in which the cuff 3 is attached to the upper arm of the user. The cuff 3 according to the present example also has a configuration in which a fluid bag (not illustrated) and a curler (not illustrated) are accommodated inside a bag-shaped exterior portion 30 having the outer surface 31 and the inner surface 32.

A hook portion 313 of a surface fastener composed of hooks and loops is formed on the outer surface 31 of the exterior portion 30, and a loop portion 321 of the surface fastener is formed on the inner surface 32 of the exterior portion 30. Therefore, when the cuff 3 is attached, the hook portion 313 and the loop portion 321 are fixed by winding the cuff 3 such that the loop portion 321 covers the hook portion 313. A nipple 314 for taking air into and out of the fluid bag is formed outside the exterior portion 30.

On the outer surface 31 of the exterior portion 30, scales 311a and 311b are provided near one end side in the circumferential direction, and a marker 312a corresponding to the scale 311a and a marker 312b corresponding to the scale 311b are provided near the other end side. The markers 312a and 312b are provided at positions shifted in the circumferential direction, reflecting the taper of the measurement target portion in the width direction (that is, the difference in the length of the arm circumference between both end portions of the cuff 3 in the widthwise direction) when the cuff 3 is attached.

As illustrated in FIGS. 8(A) and 8(B), the exterior portion 30 of the cuff 3 is tapered such that the widths of the portions near the scales 311a and 311b become narrower toward the circumferential end portions. Accordingly, as illustrated in FIG. 9, in a state where the cuff 3 is attached, the markers 312a and 312b can indicate the corresponding scales 311a and 311b without being hidden. Further, as described above, the markers 312a and 312b are provided to be shifted in the circumferential direction, reflecting the taper of the measurement target portion in the width direction. Thus, when the cuff 3 is actually attached, both the markers 312a and 312b point at the scales for the same size.

### Example 4

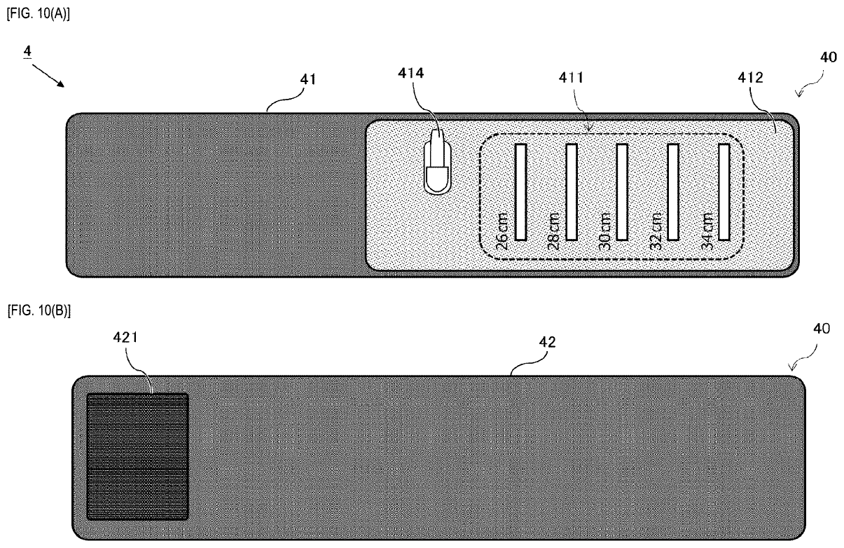

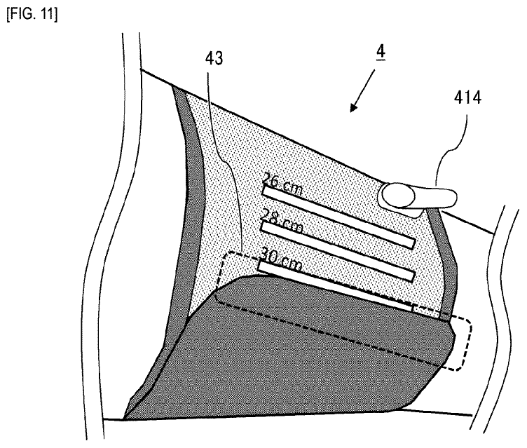

Next, a cuff 4 according to still another example will be described with reference to FIGS. 10(A), 10(B), and 11. FIG. 10(A) is a plan view of a surface (outer surface 41) of the cuff 4 that becomes an outer side when being attached, and FIG. 10(B) is a plan view of a surface (inner surface 42) of the cuff 4 that becomes an inner side when being attached. FIG. 11 is a diagram schematically illustrating a state in which the cuff 4 is attached to the upper arm of the user. The cuff 4 according to the present example also has a configuration in which a fluid bag (not illustrated) and a curler (not illustrated) are accommodated inside a bag-shaped exterior portion 40 having the outer surface 41 and the inner surface 42, and a nipple 414 for taking air into and out of the fluid bag is formed on the outside of the exterior portion 40.

A loop portion 412 of a surface fastener composed of hooks and loops is formed on the outer surface 41 of the exterior portion 40, and a hook portion 421 of the surface fastener is formed on the inner surface 42 of the exterior portion 40. When the cuff 4 is attached, the hook portion 421 and the loop portion 412 are fixed by winding the circumferential end portion on the side with the hook portion 421 and engaging the circumferential end portion with the loop portion 412. Therefore, when the cuff 4 is attached, the circumferential end portion on the side with the hook portion 421 is disposed on the outermost side (distal end). Hereinafter, the circumferential end portion on the side with the hook portion 421 will also be referred to as a distal end portion 43.

In the present example, a scale 411 is provided in the loop portion 412 of the surface fastener. Therefore, as illustrated in FIG. 11, when the hook portion 421 is engaged with the loop portion 412 at the time of attachment of the cuff 4, the distal end portion 43 is fixed while pointing at the scale. In attaching the cuff 4 to the measurement target portion, the user winds and fixes the cuff 4 such that the distal end portion 43 always comes at the same scale position, thereby preventing a situation in which the degree of winding of the cuff varies (tight or loose) every time blood pressure measurement is performed.

### Others

The description of the examples above is merely illustrative of the present invention, and the present invention is not limited to the specific examples described above. Various modified examples and combinations may be made within the scope of the technical idea of the present invention. For example, the markers indicating the scales in Examples 2 and 3 may not be dedicated markers, and marks or structures provided on the cuff for another purpose (for example, marks for artery alignment) may be used as the markers.

The marks written together with the scales are not limited to numbers or the length of the arm circumference, and may be symbols, alphabet characters, or figures. The marks are only required to be able to align predetermined portions with the same positions each time.

In the above examples, the cuff used by being attached to the upper arm has been described as an example. However, the cuff according to the present invention can also be used at another site (for example, the wrist, the thigh, or the like) as the measurement target portion. Further, the blood pressure measurement device using the cuff described in each example is also included in the technical scope of the present invention.

### REFERENCE SIGNS LIST

1, 2, 3, 4, 100 . . . Cuff
10, 20, 30, 40 . . . Exterior portion
11, 21, 31, 41 . . . Outer surface
12, 22, 32, 42 . . . Inner surface
13 . . . Fluid bag
14 . . . Folded-back portion
15 . . . Measure
23 . . . Curler
43 . . . Distal end portion
111 . . . Fold-back ring
112, 213, 313, 421 . . . Hook portion
113, 221, 321, 412 . . . Loop portion
114, 214, 314, 414 . . . Nipple
121, 141, 211, 311a, 311b, 411, 151 . . . Scale
212, 312a, 312b . . . Marker

1. A cuff to be wrapped and attached around a measurement target portion for blood pressure measurement, the cuff comprising:
an exterior portion having a bag shape, a circumferential direction that is a winding direction around the measurement target portion and a width direction that is a direction orthogonal to the circumferential direction, the exterior portion including an outer surface that is on an outer side and an inner surface that is on an inner side when the cuff is attached;
a fluid bag accommodated in the exterior portion; and
a surface fastener provided on a surface of the exterior portion, wherein
a scale that divides the exterior portion in the circumferential direction is provided in each of regions including both end portions in the width direction on the surface of the exterior portion,
the exterior portion is provided with a marker indicating a position corresponding to each of the scales,
the marker corresponding to the scale at one end portion in the width direction and the marker corresponding to the scale at the other end portion are provided at different positions in the circumferential direction, reflecting a taper of the measurement target portion in the width direction, and
the cuff is configured to be able to specify where a predetermined portion of the cuff is positioned on the scale when the cuff is attached.

2. The cuff according to claim 1, wherein
the scale is provided in a region including a central portion in the width direction.

3. The cuff according to claim 1, further comprising:
a fold-back ring configured to fold back through an outer end portion that is one end portion of the exterior portion in the circumferential direction and is exposed to outside when the cuff is attached, wherein
the scale is provided on the inner surface, and
the predetermined portion is a fold-back portion of the exterior portion when the cuff is attached.

4. The cuff according to claim 1, wherein
the exterior portion further accommodates a curler having an elastic force and configured to assist in maintaining the cuff in an annular shape,
the scale is provided on the outer surface, and
the predetermined portion is the circumferential end portion of the exterior portion that is exposed to outside when the cuff is attached.

5. The cuff according to claim 1, wherein
the exterior portion further accommodates a curler having an elastic force and configured to assist in maintaining the cuff in an annular shape,
the scale is provided on the outer surface, and
the predetermined portion is a marker provided on the outer surface.

6. A blood pressure measurement device comprising:
the cuff according to claim 1; and
a sphygmomanometer body connected to the cuff.

7. A blood pressure measurement device comprising:
the cuff according to claim 2; and
a sphygmomanometer body connected to the cuff.

8. A blood pressure measurement device comprising:
the cuff according to claim 3; and
a sphygmomanometer body connected to the cuff.

9. A blood pressure measurement device comprising:
the cuff according to claim 4; and
a sphygmomanometer body connected to the cuff.

6. A blood pressure measurement device comprising:
the cuff according to claim 5; and
a sphygmomanometer body connected to the cuff.
